(19) United States
(12) Patent Application Publication
(10) Pub. No.: US 2005/0014983 A1
(43) Pub. Date: Jan. 20, 2005

(54) Process for producing linear alpha olefins

(76) Inventors: Eric Johannes Maria De Boer, Amsterdam (NL); Harry Van Der Heijden, Amsterdam (NL); Eric Kragtwijk, Amsterdam (NL); Quoc An On, Amsterdam (NL); Johan Paul Smit, Amsterdam (NL); Arie Van Zon, Amsterdam (NL)

Correspondence Address: Donald F. Haas;Shell Oil Company, Legal - Intellectual Property, P. O. Box 2463, Houston, TX, 77252-2463, US

(21) Appl. No.: 10/883,600

(22) Filed: Jul. 1, 2004

(30) Foreign Application Priority Data
Jul. 7, 2003 (EP) 03254303.5

(51) Int. Cl. C07C 2/26; B01J 31/00

(52) U.S. Cl. 585/511; 502/102; 502/103; 502/118; 502/123

(57) ABSTRACT

A process for the production of alpha-olefins comprising reacting ethylene under oligomerization conditions in the presence of a mixture comprising: (a) a metal salt based on Fe(II), Fe(III), Co(II) or Co(III); (b) a pyridine bis-imine ligand; and (c) a co-catalyst which is the reaction product of water with one or more organometallic aluminium compounds, wherein the one or more organometallic aluminium compounds is selected from: (i) βδ-branched compounds of formula (I):
Al(CH₂—CR¹R²—CH₂—CR⁴R⁵R⁶)ₓR³_{y}H_{z}; (ii) βγ-branched compounds of formula (II)
Al(CH₂—CR¹R²—CR⁴R⁵R⁶)ₓR³_{y}H_{z} and mixtures thereof; wherein when the metal salt and the bis-arylimine pyridine ligand are mixed together they are soluble in aliphatic or aromatic hydrocarbon solvent.

### FIELD OF THE INVENTION

The present invention relates to a process for producing linear alpha olefins by ethylene oligomerization and to catalyst systems for use in said process.

### BACKGROUND OF THE INVENTION

Various processes are known for the production of higher linear alpha olefins (for example D. Vogt, Oligomerization of ethylene to higher α-olefins in Applied Homogeneous Catalysis with Organometallic Compounds Ed. B. Cornils, W. A. Herrmann, 2^{nd} Edition, Vol. 1, Ch. 2.3.1.3, page 240-253, Wiley-VCH 2002). These commercial processes afford either a Poisson or Schulz-Flory oligomer product distribution.

In order to obtain a Poisson distribution, no chain termination must take place during oligomerization. However, in contrast, in a Schulz-Flory process, chain termination does occur and is independent from chain length. The Ni-catalysed ethylene oligomerization step of the Shell Higher Olefins Process (SHOP) is a typical example of a Schulz-Flory process.

In a Schulz-Flory process, a wide range of oligomers are typically made in which the fraction of each olefin can be determined by calculation on the basis of the so-called K-factor. The K-factor, which is indicative of the relative proportions of the product olefins, is the molar ratio of [Cₙ₊₂]/[Cn] calculated from the slope of the graph of log [Cₙ mol %] versus n, where n is the number of carbon atoms in a particular product olefin. The K-factor is by definition the same for each n. By ligand variation and adjustment of reaction parameters, the K-factor can be adjusted to higher or lower values. In this way, the process can be operated to produce a product slate with an optimised economic benefit.

Since demand for the C₆-C₁₈ fraction is much higher than for the C_{>20} fraction, processes are geared to produce the lower carbon number olefins. However, the formation of the higher carbon number olefins is inevitable, and, without further processing, the formation of these products is detrimental to the profitability of the process. To reduce the negative impact of the higher carbon number olefins and of the low value C₄ fraction, additional technology has been developed to reprocess these streams and convert them into more valuable chemicals such as internal C₆-C₁₈ olefins, as is practiced in the Shell Higher Olefins Process.

However, this technology is expensive both from an investment and operational point of view and consequently adds additional cost. Therefore considerable effort is directed to keep the production of the higher carbon numbered olefins to the absolute minimum, i.e. not more than inherently associated with the Schulz-Flory K-factor.

In this regard a number of published patent applications describe catalyst systems for the polymerization or oligomerization of 1-olefins, in particular ethylene, which contain nitrogen-containing transition metal compounds. See, for example, the following patent applications which are incorporated herein by reference in their entirety: WO 92/12162, WO 96/27439, WO 99/12981, WO 00/50470, WO 98/27124, WO 99/02472, WO 99/50273, WO 99/51550, EP-A-1,127,987, WO 02/12151, WO 02/06192, WO 99/12981, WO 00/24788, WO 00/08034, WO 00/15646, WO 00/20427 and WO 01/58874 and WO03/000628.

In particular, recently published Shell applications WO01/58874, WO02/00339, WO02/28805 and WO03/011876, all of which are incorporated herein by reference in their entirety, disclose novel classes of catalysts based on bis-imine pyridine iron dichloride complexes which are highly active in the oligomerization of olefins, especially ethylene and which produce linear alpha olefins in the C₆-C₃₀ range with a Schulz-Flory distribution, said linear alpha olefins being of high purity.

It is known to use a co-catalyst such as an aluminium alkyl or aluminoxane (the reaction product of water and an aluminium alkyl) in order to activate olefin oligomerization catalysts. One such co-catalyst is MAO, i.e. methyl aluminoxane. Another such co-catalyst is MMAO, i.e. methyl aluminoxane modified by isobutyl groups.

However, during ethylene oligomerization experiments in paraffin solvents using bis-arylimine pyridine iron dichloride complexes and MMAO as co-catalyst, catalyst lifetimes have been found to be relatively low with concomitant formation of precipitates over time, despite application of an inert gas cap. Such catalyst decay is especially inconvenient during continuous operation of an ethylene oligomerization plant since precise dosing of these catalyst “solutions” or rather “ever-changing suspensions or slurries” becomes a difficult task.

One solution to this problem would be to dose the MMAO solution and the bis-arylimine pyridine iron dichloride complex solution separately and mix these streams in the ethylene oligomerization reactor. This option is unfortunately impeded however by the low solubility of the bis-arylimine pyridine iron dichloride complexes in aromatic and especially in aliphatic solvent.

Another solution to the problem of imprecise catalyst dosing would be to prepare the catalyst system in situ, i.e. within the ethylene oligomerization reactor, in such a way that the components of the catalyst system form a clear and stable solution in the aliphatic or aromatic hydrocarbon solvent used in the oligomerization reaction.

Chemtech, July 1999, pages 24-28, “Novel, highly active iron and cobalt catalysts for olefin polymerisation” by Alison Bennett, discloses that a mixture of Co(acac)₂, pyridine bis-imine ligand, and methyl alumoxane will polymerise ethylene in high yield to form a similar polyethylene product as that formed from the precatalyst complex and methylalumoxane.

It has been observed by the present inventors that Fe(III) (2,4-pentanedionate)₃, designated hereinafter as Fe(acac)₃, which is sparingly soluble in aliphatic solvents such as isooctane or heptane is transformed into a clear and stable solution by addition of an approximately equimolar amount of the appropriate bis-arylimine pyridine ligand. This allows the in-situ preparation of a Fe(III)bis-arylimine pyridine complex in the oligomerization reactor.

Use of MMAO as catalyst activator in the above-mentioned in-situ preparation gives a high initial activity of catalyst, however, catalyst lifetime is relatively short, particularly at elevated temperatures in aliphatic solvents. This is a particular problem in a continuous ethylene oligomerization plant where the temperatures are ideally above 70° C., preferably from 80-120° C., in order to avoid plugging of high molecular weight (>C20) alpha olefins in the reactor and when operating at high alpha olefin concentrations in aliphatic solvents.

Therefore, there is a need to identify alternative co-catalysts in the in-situ preparation of Fe-based catalyst systems, in order to improve catalyst lifetime. Importantly, this boost in catalyst lifetime should not be at the expense of alpha-olefin yield and purity.

It has now surprisingly been found that the use of selected βγ- and/or βδ-branched aluminium alkyl or aluminoxane co-catalysts in the in-situ preparation of bis-imine pyridine Fe and Co complexes provides catalyst systems with longer lifetimes and higher catalytic activity. At the same time, the alpha-olefin purity and alpha-olefin yield of the final product is on a par with those obtained with MMAO.

U.S. Pat. No. 6,395,668 discloses a catalyst system for the polymerisation of olefins comprising the product obtainable by contacting (a) one or more compounds of a Group 8-11 transition metal, and (b) a reaction product of water with one or more organometallic aluminium compounds. All of the ethylene polymerisation examples therein make use of a bis-imine pyridine iron precatalyst complex. There is no disclosure in this document of the preparation of linear alpha olefins using a catalyst system where the bis-imine pyridine iron complex has been prepared in-situ.

### SUMMARY OF THE INVENTION

The present invention provides a process for the preparation of alpha-olefins comprising reacting ethylene under oligomerization conditions in the presence of a mixture comprising:
(a) a metal salt based on Fe(II), Fe(III), Co(II) or Co(III);
(b) a bis-arylimine pyridine ligand; and
(c) a co-catalyst which is the reaction product of water with one or more organometallic aluminium compounds selected from:
   (i) βδ-branched compounds of formula (I):
      Al(CH₂—CR¹R²—CH₂—CR⁴R⁵R⁶)ₓR³_{y}H_{z};
      wherein R¹ is a linear or branched, saturated or unsaturated C₁-C₂₀ alkyl, C₃-C₂₀ cycloalkyl, C₆-C₂₀ aryl, C₇-C₂₀ alkylaryl radical; R² is hydrogen or a linear or branched, saturated or unsaturated C₁-C₂₀ alkyl, C₆-C₂₀ aryl, C₇-C₂₀ alkylaryl or arylalkyl radical; R³ is a linear or branched, saturated or unsaturated C₁-C₂₀ alkyl, C₃-C₂₀ cycloalkyl, C₆-C₂₀ aryl, C₇-C₂₀ alkylaryl or C₇-C₂₀ arylalkyl radical; x is an integer of from 1-3; z is 0 or 1; and y is 3-x-z; R⁴ and R⁵, the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl radicals; the substituents R¹ and R⁴ or R⁴ and R⁵ optionally form one or two rings, having 3 to 6 carbon atoms; R⁶ is hydrogen or has the same meaning of R⁴ and R⁵;
   (ii) βγ-branched compounds of formula (II)
      Al(CH₂—CR¹R²—CR⁴R⁵R⁶)ₓR³_{y}H_{z}
      wherein R¹, R², R³, R⁴, R⁵, R⁶, x, y and z are as defined hereinabove in relation to formula (I);
and mixtures thereof;
   wherein when the metal salt and the bis-arylimine pyridine ligand are mixed together they are soluble in aliphatic or aromatic hydrocarbon solvent.

In a further aspect of the present invention there is provided a catalyst system obtainable by the in-situ mixing of:
(a) a metal salt based on Fe(II), Fe(III), Co(II) or Co(III);
(b) a bis-arylimine pyridine ligand; and
(c) a co-catalyst which is the reaction product of water with one or more organometallic aluminium compounds selected from:
   (i) βδ-branched compounds of formula (I):
      Al(CH₂—CR¹R²—CH₂—CR⁴R⁵R⁶)ₓR³_{y}H_{z};
      wherein R¹ is a linear or branched, saturated or unsaturated C₁-C₂₀ alkyl, C₃-C₂₀ cycloalkyl, C₆-C₂₀ aryl, C₇-C₂₀ alkylaryl radical; R² is hydrogen or a linear or branched, saturated or unsaturated C₁-C₂₀ alkyl, C₆-C₂₀ aryl, C₇-C₂₀ alkylaryl or arylalkyl radical; R³ is a linear or branched, saturated or unsaturated C₁-C₂₀ alkyl, C₃-C₂₀ cycloalkyl, C₆-C₂₀ aryl, C₇-C₂₀ alkylaryl or C₇-C₂₀ arylalkyl radical; x is an integer of from 1 to 3; z is 0 or 1; and y is 3-x-z; R⁴ and R⁵, the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl groups; the substituents R¹ and R⁴ or R⁴ and R⁵ optionally form one or two rings, having 3 to 6 carbon atoms; R⁶ is hydrogen or has the same meaning of R⁴ and R⁵;
   (ii) βγ-branched compounds of formula (II)
      Al(CH₂—CR¹R²—CR⁴R⁵R⁶)ₓR³_{y}H_{z}
      wherein R¹, R², R³, R⁴, R⁵, R⁶, x, y and z are as defined hereinabove in relation to formula (I);
and mixtures thereof;
   wherein when the metal salt and the bis-arylimine pyridine ligand are mixed together they are soluble in aliphatic or aromatic hydrocarbon solvent.

### DETAILED DESCRIPTION OF THE INVENTION

A first essential component of the catalyst system herein is a metal salt based on Fe(II), Fe(III), Co(II) or Co(III).

The metal salt and the bis-arylimine pyridine ligand are chosen herein such that when they are mixed together they are soluble in aliphatic or aromatic hydrocarbon solvent. Ethylene oligomerization reactions are typically carried out in an aliphatic or aromatic hydrocarbon solvent.

As used herein the term “when the metal salt and the bis-arylimine pyridine ligand are mixed together they are soluble in aliphatic or aromatic hydrocarbon solvent” means that the metal salt when mixed together with the bis-arylimine pyridine ligand in a molar ratio of 1:1.2 has a solubility in heptane at 25° C. in the range of 2 ppb to 200 ppm, preferably from 2 ppm to 200 ppm and more preferably from 20 ppm to 200 ppm (wt/wt based on metal in solution). As an example, a mixture of 37 mg of Fe(acac)₃ and 57.5 mg of the bis-arylimine pyridine Ligand A prepared in the examples hereinbelow (i.e. a mixture of metal salt and bis-arylimine pyridine ligand in a molar ratio of 1:1.2) forms a substantially clear solution in 169 g of pure heptane at 25° C. (representing 35 ppm (wt/wt) of Fe (metal) in the heptane solution.

If such a mixture forms a substantially clear solution in heptane, then it should also form a substantially clear solution in other aliphatic or aromatic hydrocarbon solvents typically used in ethylene oligomerization reactions.

As used herein the term “substantially clear solution” means a visually transparent solution which does not give rise to sedimentation over time at room temperature. The term “substantially clear solution” as used herein is intended to encompass both real solutions (which contain dissolved particles with an average particle diameter of from 0.1 to 1 nm which cannot be detected by microscopic or ultramicroscopic techniques and cannot be separated by (ultra)filtration or dialysis) and colloidal solutions (which have particles with an average particle size of from 0.1 to 0.001 μm (=1 nm) which do not show sedimentation over time at room temperature).

It should be noted that within the ambit of the present invention it is possible to use a metal salt, which, when taken on its own, is insoluble or only sparingly soluble in aliphatic or aromatic solvent, provided that when it is mixed with an appropriate bis-arylimine pyridine ligand, the mixture is soluble in aliphatic or aromatic solvent.

Non-limiting examples of suitable metal salts include carboxylates, carbamates, alkoxides, thiolates, catecholates, oxalates, thiocarboxylates, tropolates, phosphinates, acetylacetonates, iminoacetylacetonates, bis-iminoacetylacetonates, the solubility of which can be tuned by an appropriate choice of substituents, as well known to those skilled in the art.

Preferred metal salts for use herein are the optionally substituted acetylacetonates, also designated as x,(x+2)-alkanedionates, such as 2,4-alkanedionates and 3,5-alkanedionates. When the acetylacetonates are substituted, preferred substituents are C₁-C₆ alkyl groups, especially methyl. Examples of suitable acetylacetonates include 2,4-pentanedionates, 2,2,6,6-tetramethyl-3,5-heptanedionates, 1-phenyl-1,3-butanedionates and 1,3-diphenyl-1,3-propanedionates. Preferred acetylacetonates for use herein are the 2,4-pentanedionates.

Metal salts based on Fe(III) are particularly preferred for use herein.

A particularly preferred metal salt for use herein is Fe(III) (2,4-pentanedionate)₃, designated herein as Fe(acac)₃. It should be noted that, on its own, Fe(acac)₃ is only sparingly soluble in aliphatic hydrocarbon solvent, but that when an appropriate bis-arylimine pyridine ligand is added, a substantially clear solution is formed in aliphatic hydrocarbon solvent.

A second essential component of the catalyst system herein is a bis-arylimine pyridine ligand.

As discussed above in relation to the metal salt, the ligand is chosen such when the metal salt and the bis-arylimine pyridine ligand are mixed together they are soluble in aliphatic or aromatic hydrocarbon solvent, as defined above.

Particularly suitable bisarylimine pyridine ligands for use herein include those having the formula (III) below: 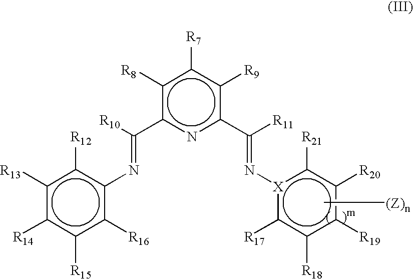
wherein X is carbon or nitrogen,
n is 0 or 1,
m is 0 or 1,
Z is a π-coordinated metal fragment,
R₇-R₁₁, R₁₃-R₁₅ and R₁₈-R₂₀ are each, independently, hydrogen, optionally substituted hydrocarbyl, an inert functional group, or any two of R₇-R₉, R₁₃-R₁₅ and R₁₈-R₂₀ vicinal to one another taken together may form a ring; R₁₂ is hydrogen, optionally substituted hydrocarbyl, an inert functional group, or taken together with R₁₃ or R₁₀ to form a ring; R₁₆ is hydrogen, optionally substituted hydrocarbyl, an inert functional group, or taken together with R₁₅ or R₁₀ to form a ring;
R₁₇ is hydrogen, optionally substituted hydrocarbyl, an inert functional group, or taken together with R₁₁ or R₁₈ to form a ring; and R₂₁ is hydrogen, optionally substituted hydrocarbyl, an inert functional group, or taken together with R₁₁ or R₂₀ to form a ring.

In relation to formula (III) above certain terms are used as follows:

The term “π-coordinated metal fragment” in relation to the group Z means that the Z group together with the ring containing the X atom represents a metallocene moiety or a sandwich or metal-arene complex which can be optionally substituted. The Z group contains a metal atom which is π-coordinated to the aromatic ring containing the X atom. The Z group can also contain one or more ligands which are coordinated to the metal atom, such as, for example (CO) ligands, such that the Z group forms the metal fragment Fe(CO)ₓ. Preferably, however, the Z group contains an optionally substituted aromatic ring which is n-coordinated to the metal. Said optionally substituted aromatic ring can be any suitable monocyclic or polycyclic, aromatic or heteroaromatic ring having from 5 to 10 ring atoms, optionally containing from 1 to 3 heteroatoms selected from N, O and S.

Preferably the aromatic ring is a monocyclic aromatic ring containing from 5 to 6 carbon atoms, such as phenyl and cyclopentadienyl. Non-limiting examples of combinations of aromatic hydrocarbon rings containing an X atom and π-coordinated metal fragments include ferrocene, cobaltocene, nickelocene, chromocene, titanocene, vanadocene, bis-benzene chromium, bis-benzene titanium and similar heteroarene metal complexes, mono-cationic arene manganese tris carbonyl, arene ruthenium dichloride.

The term “Hydrocarbyl group” in relation to the R⁷ to R²¹ groups of formula (III) above means a group containing only carbon and hydrogen atoms. Unless otherwise stated, the number of carbon atoms is preferably in the range from 1 to 30, especially from 1 to 6. The hydrocarbyl group may be saturated or unsaturated, aliphatic, cycloaliphatic or cycloaromatic, but is preferably aliphatic. Suitable hydrocarbyl groups include primary, secondary and tertiary carbon atom groups such as those described below.

The phrase “optionally substituted hydrocarbyl” in relation to the R⁷ to R²¹ groups of formula (III) above is used to describe hydrocarbyl groups optionally containing one or more “inert” heteroatom-containing functional groups. By “inert” is meant that the functional groups do not interfere to any substantial degree with the oligomerization process. Non-limiting examples of such inert groups are fluoride, chloride, silanes, stannanes, ethers, alkoxides and amines with adequate steric shielding, all well-known to those skilled in the art. Some examples of such groups include methoxy and trimethylsiloxane. Said optionally substituted hydrocarbyl may include primary, secondary and tertiary carbon atom groups of the nature described below.

The term “inert functional group” in relation to the R⁷ to R²¹ groups of formula (III) above means a group other than optionally substituted hydrocarbyl which is inert under the oligomerization process conditions herein. By “inert” is meant that the functional group does not interfere to any substantial degree with the oligomerization process. Examples of inert functional groups suitable for use herein include halide, ethers, and amines such as tertiary amines, especially fluorine and chlorine.

The term “Primary carbon atom group” as used herein means a —CH₂—R group wherein R is selected from hydrogen, an optionally substituted hydrocarbyl or an inert functional group. Examples of suitable primary carbon atom groups include, but are not limited to, —CH₃, —C₂H₅, —CH₂Cl, —CH₂OCH₃, —CH₂N(C₂H₅)₂ and —CH₂Ph. Preferred primary carbon atom groups for use herein are those wherein R is selected from hydrogen or a C₁-C₆ unsubstituted hydrocarbyl, preferably wherein R is hydrogen or a C₁-C₃ alkyl.

The term “Secondary carbon atom group” as used herein means a —CH(R)₂ group wherein R is selected from optionally substituted hydrocarbyl or an inert functional group. Alternatively, the two R groups may together represent a double bond moiety, e.g. ═CH₂, or a cycloalkyl group. Examples of secondary carbon atom groups include, but are not limited to, —CH(CH₃)₂, —CHCl₂, —CHPh₂, —CH═CH₂ and cyclohexyl. Preferred secondary carbon atom groups for use herein are those in which R is a C₁-C₆ unsubstituted hydrocarbyl, preferably a C₁-C₃ alkyl.

The term “Tertiary carbon atom group” as used herein means a —C(R)₃ group wherein each R is independently selected from an optionally substituted hydrocarbyl or an inert functional group. Alternatively, the three R groups may together represent a triple bond moiety, e.g. —C≡CPh, or a ring system containing tertiary carbon atoms such as adamantyl derivatives. Examples of tertiary carbon atom groups include, but are not limited to, —C(CH₃)₃, —CCl₃, —C≡CPh, 1-Adamantyl and —C(CH₃)₂(OCH₃). Preferred tertiary carbon atom groups for use herein are those wherein each R is a C₁-C₆ unsubstituted hydrocarbyl group, preferably wherein each R is a C₁-C₃ alkyl group, preferably wherein each R is methyl. In the case wherein each R is a methyl group, the tertiary carbon atom group is tert-butyl.

It will be appreciated by those skilled in the art that within the boundary conditions hereinbefore described, substituents R₇-R₂₁ may be readily selected to optimise the performance of the catalyst system and its economical application.

A preferred bisarylimine pyridine ligand for use herein is a ligand of formula (III) wherein X is C, m is 1 and n is 0 such that the ring containing the X atom is a 6-membered aromatic group.

Another preferred bisarylimine pyridine ligand for use herein is a ligand of formula (III) wherein X is C, m is 0, n is 1, and the ring containing X together with the Z group is a metallocene group.

Yet another preferred bisarylimine pyridine ligand for use herein is a ligand of formula (III) wherein X is N, m is 0, n is 0, such that the ring containing the X atom is a 1-pyrrolyl group.

To restrict the products to oligomers it is preferred that no more than one of R₁₂, R₁₆, R₁₇ and R²¹ is a tertiary carbon atom group. It is also preferred that not more than two of R₁₂, R₁₆, R₁₇ and R₂₁ is a secondary carbon atom group.

Preferred ligands for use herein include those of formula (III) with the following ortho substituents:
(i) R₁₂, R₁₆, R₁₇ and R₂₁ are each, independently, F or Cl;
(ii) R₁₂ and R₁₆ are primary carbon atom group, R₁₇ is H or F and R₂₁ is H, F or primary carbon atom group;
(iii) R¹² and R₁₆ are each, independently, H or F, R₁₇ and R²¹ are each, independently, F, Cl or primary carbon atom group;
(iv) R₁₂ is H or F, R₁₆ is H, F or primary carbon atom group, R₁₇ and R₂₁ are primary carbon atom groups;
(v) R₁₂ is a primary or secondary carbon atom group, R₁₆ is hydrogen, R¹⁷ and R₂₁ are H, F, Cl, primary or secondary carbon atom groups;
(vi) R₁₂ is tertiary carbon atom group, R₁₆ is hydrogen, R₁₇ is H, F, Cl, primary carbon atom group and R₂₁ is H or F;
(vii) R₁₂ is tertiary carbon atom group, R₁₆ is primary carbon atom group, R₁₇ and R₂₁ are H or F;
(viii) R₁₂ and R₁₆ are H, F, Cl, primary carbon atom group, secondary carbon atom group, R₁₇ is primary or secondary carbon atom group and R₂₁ is H;
(ix) R₁₂ is H, F, Cl, R₁₆ is H, F, Cl or primary carbon atom group, R₁₇ is tertiary carbon atom group and R₂₁ is H;
(x) R₁₂ and R₁₆ are H, F or Cl, R₁₇ is tertiary carbon atom group, R₂₁ is primary carbon atom group.

Particularly preferred ligands for use herein include those of formula (III) wherein R₇-R₉ are hydrogen and R₁₀ and R₁₁ are methyl, H, benzyl or phenyl, preferably methyl.

Especially preferred ligands for use herein include:
a ligand of formula (III), wherein R₇-R₉ are hydrogen; R₁₀ and R₁₁ are methyl; R₁₂ and R₁₆ are methyl; R₁₄ is methyl or hydrogen, R₁₃ and R₁₅ are hydrogen; R₁₇ and R₂₁ are hydrogen; R₁₈, R₁₉, and R₂₀ are independently hydrogen, methyl, or tert-butyl; X is C, m is 1, n is 0;
a ligand of formula (III), wherein R₇-R₉ are hydrogen; R₁₀ and R₁₁ are methyl; R₁₂, R₁₄ and R₁₆ are methyl; R¹³ and R₁₅ are hydrogen; R₁₇ is fluorine; and R₁₈-R₂₁ are hydrogen; and X is C, m is 1 and n is 0;
a ligand of formula (III), wherein R₇-R₉ are hydrogen; R₁₀ and R₁₁ are methyl; R₁₃-R₁₅ and R₁₈-R₂₀ are hydrogen; R₁₂, R₁₆, R₁₇ and R₂₁ are fluorine; X is C, m is 1 and n is 0;
a ligand of formula (III), wherein R₇-R₉ are hydrogen, R₁₀ and R₁₁ are methyl, R₁₂, R₁₄ and R₁₆ are methyl, R₇ and R₁₅ are hydrogen, m is 1, n is 0, X is C, R₁₇, R₁₈, R₂₀ and R₂₁ are hydrogen, R₁₉ is methoxy or trimethylsiloxy;
a ligand of formula (III), wherein R₇-R₉ are hydrogen; R₁₀ and R₁₁ are methyl; R₁₂ and R₁₆ are methyl; R₁₄ is methyl or hydrogen, R₁₃ and R₁₅ are hydrogen; R₁₇ and R₂₁ are hydrogen; R₁₈, R₁₉, and R₂₀ are independently hydrogen, methyl, or fluorine; X is C, m is 1, n is 0.

The bis-arylimine pyridine ligands for use herein can be prepared using methods well known to those skilled in the art, such as described in WO01/58874, WO02/00339, WO02/28805, WO03/011876, WO 92/12162, WO 96/27439, WO 99/12981, WO 00/50470, WO 98/27124, WO 99/02472, WO 99/50273, WO 99/51550, EP-A-1,127,987, WO 02/12151, WO 02/06192, WO 99/12981, WO 00/24788, WO 00/08034, WO 00/15646, WO 00/20427 and and WO03/000628.

A third essential component of the catalyst systems herein is a co-catalyst compound which is the reaction product of water with one or more organometallic aluminium compounds, wherein the one or more organometallic aluminium compounds is selected from:
(i) βδ-branched compounds of formula (I):
   Al(CH₂—CR¹R²—CH₂—CR⁴R⁵R⁶)ₓR³_{y}H_{z};
   wherein R¹ is a linear or branched, saturated or unsaturated C₁-C₂₀ alkyl, C₃-C₂₀ cycloalkyl, C₆-C₂₀ aryl or C₇-C₂₀ alkylaryl radical; R² is hydrogen or a linear or branched, saturated or unsaturated C₁-C₂₀ alkyl, C₆-C₂₀ aryl, C₇-C₂₀ alkylaryl or arylalkyl radical; R³ is a linear or branched, saturated or unsaturated C₁-C₂₀ alkyl, C₃-C₂₀ cycloalkyl, C₆-C₂₀ aryl, C₇-C₂₀ alkylaryl or C₇-C₂₀ arylalkyl radical; x is an integer of from 1-3; z is 0 or 1; and y is 3-x-z; R⁴ and R⁵, the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl groups; the substituents R¹ and R⁴ or R⁴ and R⁵ optionally form one or two rings, having 3 to 6 carbon atoms; R⁶ is hydrogen or has the same meaning of R⁴ and R⁵;
(ii) βγ-branched compounds of formula (II)
   Al(CH₂—CR¹R²—CR⁴R⁵R⁶)ₓR³_{y}H_{z}
   wherein R¹, R², R³, R⁴, R⁵, R⁶,x, y and z are as defined hereinabove in relation to formula (I); the substituents R¹ and R⁴ or R⁴ and R⁵ optionally form one or two rings, having 3 to 6 carbon atoms;
and mixtures thereof.

The co-catalyst compounds of formula (I) and (II) can be used in combination with other co-catalysts known in the art, such as organometallic aluminium compounds other than those having a formula (I) or (II).

Preferred co-catalysts for use herein are those prepared from compounds of formula (I) or (II) above wherein R¹ is a C₁-C₅ alkyl group, preferably C₁-C₃ alkyl, especially methyl or ethyl; R² is hydrogen or a C₁-C₅ alkyl group, preferably hydrogen; and R³ is a C₁-C₅ alkyl group.

Also preferred for use herein those co-catalysts prepared from compounds of formula (I) or (II) above wherein R⁴, R⁵ and R⁶ are independently selected from hydrogen or a C₁-C₅ alkyl, preferably independently selected from hydrogen or a C₁-C₃ alkyl.

Particularly preferred co-catalysts for use herein are those prepared from compounds of formula (I) or (II) above wherein x is 3 and z is 0.

Suitable organometallic compounds having the formula (I) include tris(2,4,4-trimethylpentyl)aluminium, bis(2,4,4-trimethylpentyl)aluminium hydride, isobutyl-bis(2,4,4-trimethylpentyl)aluminium, diisobutyl-(2,4,4-trimethylpentyl)aluminium, tris(2,4-dimethylheptyl)aluminium and bis(2,4-dimethylheptyl)aluminium hydride.

Suitable organometallic compounds having the formula (II) include tris(2,3-dimethyl-butyl)aluminium, tris(2,3,3-trimethyl-butyl)aluminium, tris(2,3-dimethyl-pentyl)aluminium, tris(2,3-dimethyl-hexyl)aluminium, tri(2,3-dimethyl-heptyl)aluminium, tris(2-methyl-3-ethyl-pentyl)aluminium, tris(2-methyl-3-ethyl-hexyl)aluminium, tris(2-methyl-3-ethyl-heptyl)aluminium, tris(2-methyl-3-propyl-hexyl)aluminium, tris(2-ethyl-3-methyl-butyl)aluminium, tris(2-ethyl-3-methyl-pentyl)aluminium, tri((2,3-diethyl-pentyl)aluminium, tris(2-propyl-3-methyl-butyl)aluminium, tris(2-isopropyl-3-methyl-butyl)aluminium, tris(2-isobutyl-3-methyl-pentyl)aluminium, tris(2,3-trimethyl-pentyl)aluminium, tris(2,3,3-trimethyl-hexyl)aluminium, tris(2-ethyl-3,3-dimethyl-butyl)aluminium, tris(2-ethyl-3,3-dimethyl-pentyl)aluminium, tris(2-isopropyl-3,3-dimethylbutyl)aluminium, tris(2-trimethylsilyl-propyl)aluminium, tris(2-methyl-3-phenyl-butyl)aluminium, tris(2-ethyl-3-phenyl-butyl)aluminium, tris(2,3-dimethyl-3-phenyl-butyl)aluminium, tris(1-menthen-9-yl)aluminium, and the corresponding compounds wherein one of the hydrocarbyl groups is replaced by hydrogen and those wherein one or more of the hydrocarbyl groups are replaced by an isobutyl group.

Particularly preferred co-catalysts for use herein are tris(2,4,4-trimethylpentyl)aluminium (designated hereinafter as “TIOAO”) and tris(2,3-dimethyl-butyl)aluminium (designated hereinafter as “TDMBAO”).

The co-catalyst compound is prepared by the addition of a suitable amount of water to the corresponding aluminium alkyl compound. The aluminium alkyl compounds can be prepared by methods known in the art and as described in WO96/02580 and WO99/21899.

The molar ratio of water to aluminium compound in the preparation of the aluminoxanes is preferably in the range from 0.01:1 to 2.0:1, more preferably from 0.02:1 to 1.2:1, even more preferably from 0.4:1 to 1:1, especially 0.5:1.

In the in-situ preparation of the catalyst systems herein, it is preferred that levels of co-catalyst and metal salt are used such that the atomic ratio of Al/Fe or Al/Co is in the range from 0.1 to 10⁶, preferably from 10 to 10⁵, and more preferably from 10² to 10⁴. It is also preferred that the molar ratio of bis-arylimine pyridine ligand/Fe or bis-aryliminepyridine ligand/Co is in the range from 10⁻⁴ to 10⁴, preferably from 10⁻¹ to 10, more preferably from 0.5 to 2, and especially 1.2.

It is possible to add further optional components to the catalyst systems herein, for example, Lewis acids and bases such as those disclosed in WO02/28805.

Oligomerization Reactions

Quantities of the catalyst components are usually employed in the oligomerization reaction mixture so as to contain from 10⁻⁴ to 10⁻⁹ gram atom of metal atom, in particular of Fe [II] or [III] metal, per mole of ethylene to be reacted.

The oligomerization reaction may be most conveniently conducted over a range of temperatures from −100° C. to +300° C., preferably in the range of from 0° C. to 200° C., and more preferably in the range of from 50° C. to 150° C.

The oligomerization reaction may be conveniently carried out at a pressure of 0.01 to 15 mPa (0.1 to 150 bar(a)), more preferably 1 to 10 mPa (10 to 100 bar(a)),. and most preferably 1.5 to 5 mPa (15 to 50 bar(a)).

The optimum conditions of temperature and pressure used for a particular catalyst system to maximise the yield of oligomer, and to minimise the competing reactions such as dimerisation and polymerisation can be readily established by one skilled in the art.

The conditions of temperature and pressure are preferably selected to yield a product slate with a K-factor within the range of from 0.40 to 0.90, most preferably in the range of from 0.60 to 0.80. In the present invention, polymerisation is deemed to have occurred when a product slate has a K-factor greater than 0.9.

The oligomerization reaction can be carried out in the gas phase or liquid phase, or mixed gas-liquid phase, depending upon the volatility of the feed and product olefins.

The oligomerization reaction is carried out in the presence of an inert hydrocarbon solvent which may also be the carrier for the catalyst components and/or feed olefin. Suitable solvents include alkanes, alkenes, cycloalkanes, and aromatic hydrocarbons. For example, solvents that may be suitably used according to the present invention include heptane, isooctane, cyclohexane, benzene, toluene, and xylene.

Reaction times of from 0.1 to 10 hours have been found to be suitable, dependent on the activity of the catalyst. The reaction is preferably carried out in the absence of air or moisture.

The oligomerization reaction may be carried out in a conventional fashion. It may be carried out in a stirred tank reactor, wherein olefin and catalyst components are added continuously to a stirred tank and reactant, product, catalyst, and unused reactant are removed from the stirred tank with the product separated and the unused reactant and optionally the catalyst recycled back to the stirred tank.

Alternatively, the reaction may be carried out in a batch reactor, wherein the catalyst precursors, and reactant olefin are charged to an autoclave, and after being reacted for an appropriate time, product is separated from the reaction mixture by conventional means, such as distillation.

After a suitable reaction time, the oligomerization reaction can be terminated by rapid venting of the ethylene in order to deactivate the catalyst system.

It is preferred that the present process is carried out in a continuous manner.

The resulting alpha olefins have a chain length of from 4 to 100 carbon atoms, preferably 4 to 30 carbon atoms, and most preferably from 4 to 20 carbon atoms.

Product olefins can be recovered suitably by distillation and further separated as desired by distillation techniques dependent on the intended end use of the olefins.

The present invention will now be illustrated by the following Examples and Figure, which should not be regarded as limiting the scope of the present invention in any way.

Experimental

General Procedures and Characterisation

All the operations with the catalyst systems were carried out under nitrogen atmosphere. All solvents used were dried using standard procedures.

Isooctane(2,4,4-trimethylpentane, 99.8% purity) was dried by prolonged nitrogen purge, followed by passing over 4 Å molecular sieves (final water content of about 1 ppm).

Anhydrous heptane (99.8% purity, ex Alrich) was dried over 4 Å molecular sieves (final water content of about 1 ppm).

Anhydrous toluene (99.8% purity) (ex. Aldrich) was dried over 4 Å molecular sieves (final water content of about 3 ppm).

Ethylene (99.5% purity) was purified over a column containing 4 Å molecular sieves and BTS catalyst (ex. BASF) in order to reduce water and oxygen content to <1 ppm.

The oligomers obtained were characterized by Gas Chromatography (GC), in order to evaluate oligomer distribution using a HP 5890 series II apparatus and the following chromatographic conditions:

Column: HP-1 (cross-linked methyl siloxane), film thickness=0.25 μm, internal diameter=0.25 mm, length 60 m (by Hewlett Packard); injection temperature: 325° C.; detection temperature: 325° C.; initial temperature: 40° C. for 10 minutes; temperature program rate: 10.0° C./minute; final temperature: 325° C. for 41.5 minutes; internal standard: n-hexylbenzene.

Response factors for the even linear α-olefins, for the internal hexenes (cis- and trans-2-hexene and cis- and trans-3-hexene) and the branched hexenes(3-methyl-1-pentene and 2-ethyl-1-butene) relative to n-hexylbenzene (internal standard) were determined using a standard calibration mixture. The response factors of the branched and internal dodecanes were assumed to be equal to the corresponding linear olefins.

The yields of the C₄-C₃₀ olefins were obtained from the GC analysis, from which the K-factor and the theoretical yield of C₄-C₁₀₀ olefins, i.e. total oligomerization product (Total Product), were determined by regression analysis, using the C₆-C₂₈ data. In the case of an almost ideal Schulz-Flory distribution (standard error of the K-factor, determined by regression analysis<0.03) and in the absence of polyethylene formation the amount of above-mentioned Total Product is assumed equal to the ethylene consumption.

The relative amounts of the linear 1-hexene amongst all hexene isomers, the relative amount of 1-dodecene amongst all dodecene isomers and the relative amount of 1-octadecene amongst all octadecene isomers found from the GC analysis is used as a measure of the selectivity of the catalyst towards linear alpha-olefin formation. The wt% data given in Table 1 on Alpha Olefin Products is quoted on this basis.

By turnover frequency (TOF) is meant the number of moles of ethylene oligomerized per hour per mole of iron compound.

The NMR data were obtained at room temperature with a Varian 300 MHz or 400 MHz apparatus.

The metal salt used for the in-situ preparation of the catalyst is Fe(III) (2,4-pentanedionate)₃, commercially available from Aldrich.

The pyridine bis-imine ligand used for the in-situ preparation of the catalyst in Examples 1-17 is 2-[1-(2,4,6-trimethylphenylimino)ethyl]-6-[1-(3,5-di-tert-butylphenylimino)ethyl]pyridine (hereinafter “Ligand A”) which was prepared according to the method below and which has the formula:

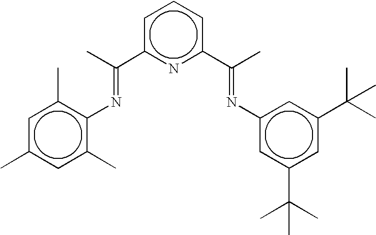

### Preparation of 2-[1-(2,4,6-trimethylphenylimino)ethyl]-6-[1-(3,5-di-tert-butylphenylimino)ethyl]pyridine

2-[1-(2,4,6-trimethylphenylimino)ethyl]-6-acetylpyridine (1.3 g, 4.64 mmol), prepared according to the method disclosed in WO02/28805, and 3,5-di-tert-butylaniline (1 g, 4.87 mmol) were dissolved in 100 ml of toluene. To this solution, 4 Å molecular sieves were added. After standing for 2 days the mixture was filtered. The solvent was removed in vacuo. The residue was washed with methanol and crystallized from ethanol.

Yield 1.1 g (51%) of 2-[1-(2,4,6-trimethylphenylimino)ethyl]-6-[1-(3,5-di-tert-butylphenylimino)ethyl]pyridine. 1H-NMR (CDCl₃) δ 8.43 (d, 1H, Py-Hₘ), 8.37 (d, 1H, Py-Hₘ), 7.87 (t, 1H, Py-Hₚ), 7.16 (t, 1H, ArH), 6.89 (s, 2H, ArH), 6.69 (d, 2H, ArH), 2.42 (s, 3H, Me), 2.29 (s, 3H, Me), 2.22 (s, 3H, Me), 2.01 (s, 6H, Me), 1.33 (s, 18H, Bu^{t}).

The pyridine bis-imine ligand used for the in-situ preparation of the catalyst in Examples 18-21 is 2,6-bis-[1-(2,6-difluorophenylimino)ethyl]pyridine (hereinafter “Ligand B”) which was prepared according to the method disclosed in WO02/00339 and which has the formula below:

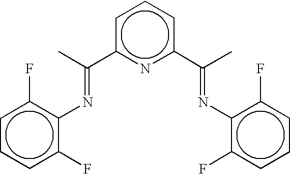

Alternatively, any of the ligands disclosed in WO02/28805, WO 02/00339, WO01/58874 or WO03/011876 could be used in the oligomerization experiments below.

The co-catalysts used in the experiments below were prepared by the addition of 0.5 mol of water to 1 mol of the corresponding aluminium alkyl in toluene at 0° C. (Note that isooctane is used as the solvent in Examples 11-19). The corresponding aluminium alkyls used in the experiments below are prepared according to the methods described in U.S. Pat. No. 6,395,668 B1 or WO99/21899 or may be purchased from commercially available sources as indicated below.

The co-catalysts used in the experiments below are as follows:
TFPPAO used in Comparative Examples 12 and 19 is prepared by the addition of 0.5 mol of water to 1 mol of tris-[2-(4-fluorophenyl)-propyl]aluminium, the latter compound being prepared according to the method disclosed in U.S. Pat. No. 6,395,668 B1.
TPPAO used in Comparative Example 15 is prepared by the addition of 0.5 mol of water to 1 mol of tris-(2-phenylpropyl)aluminium, the latter compound being prepared according to the method disclosed in U.S. Pat. No. 6,395,668 B1.
TIBAO used in Comparative Example 17 is prepared by the addition of 0.5 mol of water to 1 mol of tris-(2-methylpropyl)aluminium (or tri-isobutyl aluminium), the latter compound being commercially available from Aldrich.
TNOAO used in Comparative Example 4, 8 and 9 is prepared by the addition of 0.5 mol of water to 1 mol of tri-n-octyl aluminium, the latter compound being commercially available from Aldrich (25% wt tri-n-octyl aluminium solution in hexanes).
TDMBAO used in Examples 2, 5 and 20 is prepared by the addition of 0.5 mol of water to 1 mol of tris-(2,3-dimethylbutyl)aluminium, the latter compound being prepared according to the method disclosed in WO99/21899.
TIOAO used in Examples 3, 6 and 13 is prepared by the addition of 0.5 mol of water to 1 mol of tris-(2,4,4-trimethylpentyl)aluminium (or tri-isooctyl aluminium), the latter compound being commercially available (7.49% wt Al).from Crompton GmbH, Ernst-Schering-Str. 14, D-59192 Bergkamen, Germany.
TEA used in Comparative Example 16 is triethylaluminium which was used in its unhydrolysed form and which is commercially available from Aldrich.
MMAO used in Comparative Examples 1, 7, 10, 11, 14, 18 and 21 is modified methyl aluminoxane (MAO) wherein about 25% of the methyl groups are replaced with isobutyl groups. This was purchased as MMAO-3A in heptane ([Al]=6.42% wt) from AKZO-NOBEL Chemicals B.V., Amersfoort, The Netherlands.
   Oligomerization Experiments

### EXAMPLES 1-10

Oligomerization experiments 1-10 were carried out in a 0.5-litre stainless steel reactor. The reactor is scavenged at 70° C. using 0.15 g MMAO and 125 ml anhydrous heptane in an inert atmosphere for at least 30 minutes. After draining the contents, 125 ml of anhydrous heptane and the designated co-catalyst is added to the reactor, followed after pressurizing with ethylene to 16 bar(a) at 40° C., by addition of a mixture of the designated ligand (Ligand A) and Fe(2,4-pentanedionate)₃ (Fe added=0.25 μmol; ligand/Fe molar ratio=1.2±0.1; Al/Fe molar ratio=700±50, unless otherwise indicated). Each addition (4 ml in toluene) to the reactor by the injection system is followed by rinsing of the system with 2×4 ml of toluene. The total solvent content of the reactor after 2 additions of the catalyst components=ca. 150 ml of heptane/toluene=8/2(wt/wt)). After the initial exotherm the reactor was brought to 70° C. as swiftly as possible, whilst monitoring the temperature, pressure and ethylene uptake. When the desired ethylene consumption has been reached or the uptake falls below 0.2N litre/min, the reaction is terminated by rapid venting and subsequent draining of the product.

### EXAMPLES 11-19

Examples 11-19 are carried out in a 1-litre reactor, using isooctane as the reactor solvent, the catalyst component solvent, rinsing agent and as the solvent used to prepare the aluminoxanes. The amounts of Fe(2,4-pentanedionate)3 and solvent are twice those mentioned above for the experiments carried out in Examples 1-10 above. Hence, Fe added=0.5 μmol; total solvent content of the reactor after 2 additions of catalyst components=ca. 310 ml of isooctane. The ligand/Fe molar ratio is the same as in Examples 1-10. The Al/Fe molar ratio is 700±50, unless otherwise indicated. In Example 14 the sequence of addition of co-catalyst and ligand/Fe(2,4-pentanedionate)₃ is reversed.

### EXAMPLES 20-21

Examples 20-21 are carried out in a 1-litre reactor, using heptane as the reactor solvent and toluene as the catalyst solvent and rinsing agent; the amounts of Fe(2,4-pentanedionate)₃ and solvent are twice those used in the Examples 1-10 above. The aluminoxane co-catalyst is added in two portions, one before and one after the addition of the mixture of ligand and Fe(2,4-pentanedionate)₃. Hence, Fe added=0.5 μmol; total solvent content of the reactor after 3 additions of catalyst components=ca. 340 ml of heptane/toluene=7/3(wt/wt). The ligand/Fe molar ratio is the same as in Examples 1-10. The Al/Fe molar ratio in Examples 20 and 21 is 1700 and 1800, respectively, as indicated in Table 1.

The amount and purity of olefins were determined by gas chromatography. The data are reported in Table 1 below.

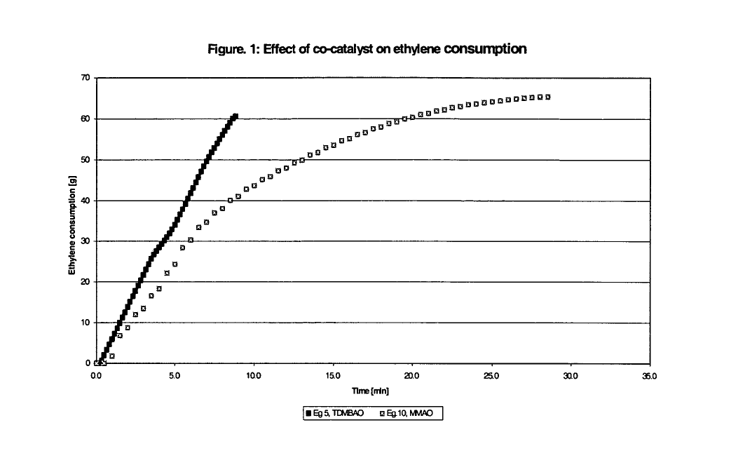

From the experimental data provided in Table 1 it can be seen that with the 2-[1-(2,4,6-trimethylphenylimino)ethyl]-6-[1-(3,5-di-tert-butylphenylimino)ethyl]pyridine ligand (Ligand A) in heptane/toluene 8/2 (wt/wt) using an Al/Fe molar ratio of 1500 the differences in turnover frequency (TOF), K-factor and α-olefin content between MMAO, TDMBAO and TIOAO are small. Only TNOAO gives a lower TOF, but a similar product distribution and product purity (see Examples 1, 2, 3, 4). At an Al/Fe ratio of 700 mol/mol, however, there is a distinct difference between the catalyst activities emerging from the various co-catalysts, as indicated by the TOF's for a given α-olefin production and by FIG. 1. It appears that TDMBAO and TIOAO (βγ- and βδ-branched co-catalysts, respectively, lying within the scope of the present invention) are better co-catalysts (higher TOFs and lower decay) than MMAO and TNOAO (co-catalysts lying outside the scope of the present invention) (See Examples 5, 6, 7 and 8). The K-factors and their standard errors—the latter being a measure of obedience of a Schulz-Flory distribution—and the α-olefin purity are on a par with those obtained with MMAO at similar final AO concentrations.

FIG. 1 shows in graphical form the comparative effects of TDMBAO and MMAO in Examples 5 and 10, respectively, on the ethylene consumption over time for an Al/Fe molar ratio of 700.

From Comparative Example 12 it can be seen that TFPPAO, a β-alkyl-β-aryl-branched aluminoxane (i.e. a ββ-branched co-catalyst lying outside the scope of the present invention), is a co-catalyst showing a high TOF and very little decay at an Al/Fe ratio of 700, i.e. after some 100 normal liters (Nl) of ethylene consumption the reaction was still running at stable uptake of 4 Nl ethylene/min. However for production of alpha olefins, TFPPAO is not such a good co-catalyst since the α-olefin purity is lower than for the other co-catalysts within the scope of the present invention at comparable Al/Fe molar ratios (see Examples 12 and 13 and Examples 5 and 6). The parent compound of TFPPAO, namely TPPAO (also a ββ-branched co-catalyst lying outside the scope of the present invention) (see Example 15), does not show any oligomerization activity at all. The same is true for the ββ-branched aluminoxane, TIBAO, and the non-hydrolysed triethyl aluminium (TEA) (see Examples 17 and 16, respectively) (both of which are co-catalysts lying outside the present invention).

It can be seen from Table 1 that the 2,6-bis-[1-(2,6-difluorophenylimino)ethyl]pyridine ligand (Ligand B) in isooctane with TFPPAO (a co-catalyst falling outside the scope of the present invention) at an Al/Fe ratio of 700, the catalyst system exhibits a high activity and very little decay, although at the expense of the α-olefin purity (see Comparative Example 19). The use of TDMBAO (a βγ-branched co-catalyst lying within the scope of the present invention) with Ligand B gives a TOF comparable to that of MMAO, but a somewhat higher α-olefin purity (compare the alpha olefin content of octadecenes fraction for Examples 20 and 21).

In summary, the results of Examples 1-21 indicate that at low Al/Fe ratios (700) the βγ-branched aluminoxane, TDMBAO, and the βε-branched aluminoxane, TIOAO, are good co-catalysts in the in-situ preparation of Fe(II) catalyst systems from the Fe(2,4-pentanedionate)₃ complex and appropriate ligand, particularly with Ligand A. In particular, they appear to be better catalysts than MMAO, TPPAO, TFPPAO, TIBAO, TNOAO and TEA (which are not βγ- or βδ-branched). The use of TDMBAO and TIOAO provides for the production of high purity alpha olefins in almost ideal Schulz-Flory distributions and low catalyst decays (high turnovers). Moreover, these co-catalysts have a high solubility and stability in paraffin solvents.

In Table 1 below the letters a-j have the following meanings:
a Reaction starts with an exotherm of less than 3° C. after heating to 60-65° C.
b TOF=Turn Over Frequency. Ethylene consumption derived from total product (C₄-C₁₀₀ olefins, as determined by regression analysis, using C₆-C₂₈ GC data), unless otherwise indicated
c Using ethylene uptake, determined by mass flow meter (from Bronkhorst High-Tech B.V., Nijverheidsstraat 1a, 7261 AK Ruurlo, The Netherlands, Type: F-201C-FA-00-Z)
d Schulz-Flory K-factor determined by regression analysis of C₆-C₂₈ GC-data
e Schulz-Flory K-factor determined by regression analysis of C₆-C₁₆ GC data
f Low, due to the presence of traces of hexanes (from TNOAO co-catalyst)
g Branched hexenes, dodecenes and octadecenes=0.5, 2.6 and 5.0 % wt; internal hexenes, dodecenes and octadecenes=0.1, 0.2 and 0.2 % wt, respectively
h Branched hexenes, dodecenes and octadecenes=1.0, 5.7 and 10.9% wt; internal hexenes, dodecenes and octadecenes=0.1, 0.2 and 0.2 % wt, respectively
i Branched hexenes, dodecenes and octadecenes=0.5, 3.2 and 6.5% wt; internal hexenes, dodecenes and octadecenes=0.1, 0.1 and 0.1 % wt, respectively

j Branched hexenes, dodecenes and octadecenes=0.7, 3.6 and 6.7 % wt; internal hexenes, dodecenes and octadecenes=0.1, 0.2 and 0.2 % wt, respectively.

TABLE 1
Batch-experiments using in-situ prepared Fe-catalysts

| Example | | Co-cat | | | TOF^{b} | | | Alpha Olefin | | |
|---|---|---|---|---|---|---|---|---|---|---|
| (Comp = | | (Al/Fe | | Final | Mol | | | Products | | |
| Comparative | Li- | molar | Ethylene | [AO] | ethylene/(Mol | K- | Std | % wt | | |
| Example) | gand | ratio) | consumption^{b} g | % wt | Fe × hour) | factor^{d} | Error^{d} | C6 | C12 | C18 |
| 1 (Comp) | A | MMAO | 62.3 | 37 | 5.9 × 10⁷ | 0.67 | 0.02 | 99.4 | 96.8 | 94.6 |
| | | (1500) | | | | | | | | |
| 2 | A | TDMBAO | 63.6 | 38 | 6.1 × 10⁷ | 0.67 | 0.02 | 99.3 | 96.4 | 94.2 |
| | | (1500) | | | | | | | | |
| 3 | A | TIOAO | 76.4 | 39 | 6.0 × 10⁷ | 0.67 | 0.02 | 99.3 | 96.2 | 93.6 |
| | | (1500) | | | | | | | | |
| 4 (Comp) | A | TNOAO | 46.0 | 29 | 1.1 × 10⁷ | 0.69 | 0.02 | 96.6^{f} | 98.0 | 96.7 |
| | | (1500) | | | | | | | | |
| 5 | A | TDMBAO | 60.8 | 36 | 5.8 × 10⁷ | 0.67 | 0.02 | 99.3 | 96.5 | 94.2 |
| | | (700) | | | | | | | | |
| 6 | A | TIOAO | 60.7 | 37 | 5.2 × 10⁷ | 0.68 | 0.03 | 99.2 | 96.2 | 93.8 |
| | | (700) | | | | | | | | |
| 7 (Comp) | A | MMAO | 53.1 | 33 | 1.8 × 10⁷ | 0.67 | 0.02 | 99.5 | 96.9 | 94.8 |
| | | (700) | | | | | | | | |
| 8 (Comp) | A | TNOAO | 66.8 | 38 | 1.9 × 10⁷ | 0.69 | 0.02 | 99.2^{f} | 97.5 | 95.8 |
| | | (700) | | | | | | | | |
| 9 (Comp) | A | TNOAO | 42.5 | 28 | 1.9 × 10⁷ | 0.67 | 0.02 | 98.3^{f} | 97.9 | 96.4 |
| | | (700) | | | | | | | | |
| 10 (Comp) | A | MMAO | 65.6 | 38 | 2.0 × 10⁷ | 0.67 | 0.02 | 99.4 | 97.1 | 95.2 |
| | | (700) | | | | | | | | |
| 11 (Comp) | A | MMAO | 69.7 | 24 | 1.1 × 10⁷ | 0.70 | 0.02 | 99.4^{g} | 97.2^{g} | 94.9^{g} |
| | | (1400) | | | | | | | | |
| 12 (Comp) | A^{a} | TFPPAO | 136.2 | 39 | 2.5 × 10⁷ | 0.67 | 0.01 | 98.8^{h} | 94.1^{h} | 88.9^{h} |
| | | (700) | | | | | | | | |
| 13 | A^{a} | TIOAO | 86.6 | 29 | 0.7 × 10⁷ | 0.66 | 0.01 | 99.5ⁱ | 96.7ⁱ | 93.5ⁱ |
| | | (700) | | | | | | | | |
| 14 (Comp) | A | MMAO | 126.2 | 37 | 1.6 × 10⁷ | 0.71 | 0.02 | 99.2^{j} | 96.2^{j} | 93.1^{j} |
| | | (1500) | | | | | | | | |
| 15 (Comp) | A | TPPAO | 0 | 0 | 0 | — | — | — | — | — |
| | | (700) | | | | | | | | |
| 16 (Comp) | A | TEA | 0 | 0 | 0 | — | — | — | — | — |
| | | (700) | | | | | | | | |
| 17 (Comp) | B | TIBAO | 0 | 0 | 0 | — | — | — | — | — |
| | | (700) | | | | | | | | |
| 18 (Comp) | B^{a} | MMAO | 73.8^{c} | 26 | 0.9 × 10^{7c} | 0.37^{e} | 0.02^{e} | 95.6 | 86.8 | 83.6 |
| | | (1700) | | | | | | | | |
| 19 (Comp) | B^{a} | TFPPAO | 123.2^{c} | 37 | 1.8 × 10^{7c} | 0.39^{e} | 0.003^{e} | 93.6 | 81.3 | 71.6 |
| | | (700) | | | | | | | | |
| 20 | B^{a} | TDMBAO | 71.5^{c} | 22 | 1.3 × 10^{7c} | 0.44^{e} | 0.02^{e} | 94.9 | 89.8 | 89.4 |
| | | (1700) | | | | | | | | |
| 21 (Comp) | B^{a} | MMAO | 71.0^{c} | 22 | 2.0 × 10^{7c} | 0.41^{e} | 0.01^{e} | 92.3 | 84.0 | 82.9 |
| | | (1800) | | | | | | | | |

1. A process for the production of alpha-olefins comprising reacting ethylene under oligomerization conditions in the presence of a mixture comprising:
(a) a metal salt based on Fe(II), Fe(III), CO(II) or Co(III);
(b) a pyridine bis-imine ligand; and
(c) a co-catalyst which is the reaction product of water with one or more organometallic aluminium compounds, wherein the one or more organometallic aluminium compounds is selected from:
(i) βδ-branched compounds of formula (I):
Al(CH₂—CR¹R²—CH₂—CR⁴R⁵R⁶)ₓR³_{y}H_{z};
wherein R¹ is a linear or branched, saturated or unsaturated C₁-C₂₀ alkyl, C₃-C₂₀ cycloalkyl, C₆-C₂₀ aryl or C₇-C₂₀ alkylaryl radical; R² is hydrogen or a linear or branched, saturated or unsaturated C₁-C₂₀ alkyl, C₆-C₂₀ aryl, C₇-C₂₀ alkylaryl or arylalkyl radical; R³ is a linear or branched, saturated or unsaturated C₁-C₂₀ alkyl, C₃-C₂₀ cycloalkyl, C₆-C₂₀ aryl, C₇-C₂₀ alkylaryl or C₇-C₂₀ arylalkyl radical; x is an integer of from 1 to 3; z is 0 or 1; and y is 3-x-z; R⁴ and R⁵, the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl radicals; the substituents R¹ and R⁴ or R⁴ and R⁵ optionally form one or two rings, having 3 to 6 carbon atoms; R⁶ is hydrogen or has the same meaning of R⁴ and R⁵;
(ii) βγ-branched compounds of formula (II)
Al(CH₂—CR¹R²—CR⁴R⁵R⁶)ₓR³_{y}H_{z}
wherein R¹, R², R³, R⁴, R⁵, R⁶, x, y and z are as defined hereinabove in relation to formula (I); R⁴ and R⁵ , the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl groups; the substituents R¹ and R⁴ or R⁴ and R⁵ optionally form one or two rings, having 3 to 6 carbon atoms; R⁶ is hydrogen or has the same meaning of R⁴ and R⁵;
and mixtures thereof;
wherein when the metal salt and the bis-arylimine pyridine ligand are mixed together they are soluble in aliphatic or aromatic hydrocarbon solvent.

2. The process of claim 1 wherein the metal salt is selected from the group consisting of carboxylates, carbamates, alkoxides, thiolates, catecholates, oxalates, thiocarboxylates, tropolates, phosphinates, acetylacetonates, iminoacetylacetonates, and bis-iminoacetylacetonates.

3. The process of claim 1 wherein the metal salt is an Fe(III) salt.

4. The process of claim 2 wherein the metal salt is an acetylacetonate.

5. The process of claim 3 wherein the metal salt is Fe(2,4-pentanedionate)₃.

6. The process of claim 1 wherein the bisarylimine pyridine ligand is selected from ligands having the formula (I) below: 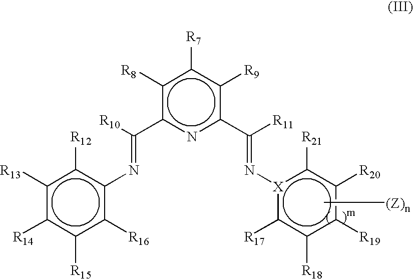
wherein X is carbon or nitrogen,
n is 0 or 1,
m is 0 or 1,
Z is a n-coordinated metal fragment,
R₇-R₁₁, R₁₃-R₁₅ and R₁₈-R₂₀ are each, independently, hydrogen, optionally substituted hydrocarbyl, an inert functional group, or any two of R₇-R₉, R₁₃-R₁₅ and R₁₈-R₂₀ vicinal to one another taken together may form a ring; R₁₂ is hydrogen, optionally substituted hydrocarbyl, an inert functional group, or taken together with R₁₃ or R₁₀ to form a ring;
R₁₆ is hydrogen, optionally substituted hydrocarbyl, an inert functional group, or taken together with R₁₅ or R₁₀ to form a ring; R₁₇ is hydrogen, optionally substituted hydrocarbyl, an inert functional group, or taken together with R₁₁ or R₁₈ to form a ring; and R₂₁ is hydrogen, optionally substituted hydrocarbyl, an inert functional group, or taken together with R₁₁ or R₂₀ to form a ring.

7. The process of claim 6 wherein R⁷-R⁹ are hydrogen;
R¹⁰ and R₁₁ are methyl; R¹² and R₁₆ are methyl; R₁₄ is methyl or hydrogen; R₁₃ and R₁₅ are hydrogen; R₁₇ and R₂₁ are hydrogen; R₁₈, R₁₉ and R₂₀ are independently hydrogen, methyl or tert-butyl; X is C, m is 1 and n is 0.

8. The process of claim 6 wherein the ligand is a ligand of formula (III) wherein X is C, m is 1 and n is 0 such that the ring containing the X atom is a 6-membered aromatic group.

9. The process of claim 6 wherein the ligand is a ligand of formula (III) wherein X is C, m is 0, n is 1, and the ring containing X together with the Z group is a metallocene group.

10. The process of claim 6 wherein the ligand is a ligand of formula (III) wherein X is N, m is 0, n is 0, such that the ring containing the X atom is a 1-pyrrolyl group.

11. The process of claim 6 wherein the ligand is a ligand of formula (III) wherein no more than one of R₁₂, R₁₆, R₁₇ and R₂₁ is a tertiary carbon atom group.

12. The process of claim 6 wherein the ligand is a ligand of formula (III) wherein not more than two of R₁₂, R₁₆, R₁₇ and R²¹ is a secondary carbon atom group.

13. The process of claim 6 wherein the ligand is a ligand of formula (III) which has the following ortho substituents: R₁₂, R₁₆, R₁₇ and R₂₁ are each, independently, F or Cl.

14. The process of claim 6 wherein the ligand is a ligand of formula (III) which has the following ortho substituents: R₁₂ and R₁₆ are primary carbon atom group, R₁₇ is H or F and R₂₁ is H, F or primary carbon atom group.

15. The process of claim 6 wherein the ligand is a ligand of formula (III) which has the following ortho substituents: R₁₂ and R₁₆ are each, independently, H or F, R₁₇ and R²¹ are each, independently, F, Cl or primary carbon atom group.

16. The process of claim 6 wherein the ligand is a ligand of formula (III) which has the following ortho substituents: R₁₂ is H or F, R₁₆ is H, F or primary carbon atom group, R₁₇ and R₂₁ are primary carbon atom groups.

17. The process of claim 6 wherein the ligand is a ligand of formula (III) which has the following ortho substituents: R₁₂ is a primary or secondary carbon atom group, R₁₆ is hydrogen, R₁₇ and R²¹ are H, F, Cl, primary or secondary carbon atom groups.

18. The process of claim 6 wherein the ligand is a ligand of formula (III) which has the following ortho substituents: R₁₂ is tertiary carbon atom group, R₁₆ is hydrogen, R¹⁷ is H, F, Cl, primary carbon atom group and R₂₁ is H or F.

19. The process of claim 6 wherein the ligand is a ligand of formula (III) which has the following ortho substituents: R₁₂ is tertiary carbon atom group, R₁₆ is primary carbon atom group, R₁₇ and R₂₁ are H or F.

20. The process of claim 6 wherein the ligand is a ligand of formula (III) which has the following ortho substituents: R₁₂ and R₁₆ are H, F, Cl, primary carbon atom group, secondary carbon atom group, R₁₇ is primary or secondary carbon atom group and R²¹ is H.

21. The process of claim 6 wherein the ligand is a ligand of formula (III) which has the following ortho substituents: R₁₂ is H, F, Cl, R₁₆ is H, F, Cl or primary carbon atom group, R₁₇ is tertiary carbon atom group and R₂₁ is H.

22. The process of claim 6 wherein the ligand is a ligand of formula (III) which has the following ortho substituents: R¹² and R₁₆ are H, F or Cl, R₁₇ is tertiary carbon atom group, R₂₁ is primary carbon atom group.

23. The process of claim 6 wherein the ligand is a ligand of formula (III) wherein R₇-R₉ are hydrogen and R₁₀ and R₁₁ are methyl, H, benzyl or phenyl.

24. The process of claim 6 wherein the ligand is a ligand of formula (III) wherein R₇-R₉ are hydrogen; R₁₀ and R₁₁ are methyl; R₁₂ and R₁₆ are methyl; R₁₄ is methyl or hydrogen, R₁₃ and R₁₅ are hydrogen; R₁₇ and R²¹ are hydrogen; R₁₈, R₁₉, and R²⁰ are independently hydrogen, methyl, or tert-butyl; X is C, m is 1, n is 0.

25. The process of claim 6 wherein the ligand is a ligand of formula (III) wherein R₇-R₉ are hydrogen; R₁₀ and R₁₁ are methyl; R₁₂, R₁₄ and R₁₆ are methyl; R₁₃ and R₁₅ are hydrogen; R₁₇ is fluorine; and R₁₈-R₂₁ are hydrogen; and X is C, m is 1 and n is 0.

26. The process of claim 6 wherein the ligand is a ligand of formula (III) wherein R₇-R₉ are hydrogen; R₁₀ and R₁₁ are methyl; R₁₃-R₁₅ and R₁₈-R₂₀ are hydrogen; R₁₂, R₁₆, R₁₇ and R₂₁ are fluorine; X is C, m is 1 and n is 0.

27. The process of claim 6 wherein the ligand is a ligand of formula (III) wherein R₇-R₉ are hydrogen, R₁₀ and R₁₁ are methyl, R₁₂, R₁₄ and R₁₆ are methyl, R₇ and R₁₅ are hydrogen, m is 1, n is 0, X is C, R₁₇, R₁₈, R₂₀ and R₂₁ are hydrogen, R₁₉ is methoxy or trimethylsiloxy.

28. The process of claim 6 wherein the ligand is a ligand of formula (III) wherein R₇-R₉ are hydrogen; R₁₀ and R₁₁ are methyl; R₁₂ and R₁₆ are methyl; R₁₄ is methyl or hydrogen, R¹³ and R₁₅ are hydrogen; R₁₇ and R²¹ are hydrogen; R₁₈, R₁₉, and R₂₀ are independently hydrogen, methyl, or fluorine; X is C, m is 1, n is 0.

29. The process of claim 1 wherein in the organometallic aluminium compounds of formulae (I) and (II) R¹ is a C₁-C₅ alkyl group; R² is hydrogen or a C₁-C₅ alkyl group; and R³ is a C₁-C₅ alkyl group.

30. The process of claim 1 wherein in the organometallic aluminium compounds of formula (I) or (II) above wherein R⁴, R⁵ and R⁶ are independently selected from hydrogen or a C₁-C₅ alkyl.

31. The process of claim 1 wherein in the organometallic aluminium compounds of formula (I) or (II) above wherein x is 3 and z is 0.

32. The process of claim 1 wherein the organometallic aluminium compound is tris(2,4,4-trimethylpentyl)aluminium.

33. The process of claim 1 wherein the organometallic aluminium compound is tris(2,3-dimethyl-butyl)aluminium.

34. A catalyst system obtainable by the in-situ mixing of
(a) a metal salt based on Fe(II), Fe(III), Co(II) or Co(III) and which is capable of being solubilized in aliphatic or aromatic solvent;
(b) a pyridine bis-imine ligand; and
(c) a co-catalyst which is the reaction product of water with one or more organometallic aluminium compounds, wherein the one or more organometallic aluminium compounds is selected from:
(i) βδ-branched compounds of formula (I):
Al(CH₂—CR¹R²—CH₂—CR⁴R⁵R⁶)ₓR³_{y}H_{z};
wherein R¹ is a linear or branched, saturated or unsaturated C₁-C₂₀ alkyl, C₃-C₂₀ cycloalkyl, C₆-C₂₀ aryl or C₇-C₂₀ alkylaryl radical; R² is hydrogen or a linear or branched, saturated or unsaturated C₁-C₂₀ alkyl, C₆-C₂₀ aryl, C₇-C₂₀ alkylaryl or arylalkyl radical; R³ is a linear or branched, saturated or unsaturated C₁-C₂₀ alkyl, C₃-C₂₀ cycloalkyl, C₆-C₂₀ aryl, C₇-C₂₀ alkylaryl or C₇-C₂₀ arylalkyl radical; x is an integer of from 1 to 3; z is 0 or 1; and y is 3-x-z; R⁴ and R⁵, the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl radicals; the substituents R¹ and R⁴ or R⁴ and R⁵ optionally form one or two rings, having 3 to 6 carbon atoms; R⁶ is hydrogen or has the same meaning of R⁴ and R⁵;
(ii) βγ-branched compounds of formula (II)
Al(CH₂—CR¹R²—CR⁴R⁵R⁶)ₓR³_{y}H_{z}
wherein R¹, R², R³, R⁴, R⁵, R⁶, x, y and z are as defined hereinabove in relation to formula (I); R⁴ and R⁵, the same or different from each other, are linear or branched, saturated or unsaturated C₁-C₂₀ alkyl, C₃-C₂₀ cycloalkyl, C₆-C₂₀ aryl, C₇-C₂₀ arylalkyl or alkylaryl groups; the substituents R¹ and R⁴ or R⁴ and R⁵ optionally form one or two rings, having 3 to 6 carbon atoms; R⁶ is hydrogen or has the same meaning of R⁴ and R⁵;
or mixtures thereof;
wherein when the metal salt and the bis-arylimine pyridine ligand are mixed together they are soluble in aliphatic or aromatic hydrocarbon solvent.

35. The catalyst system of claim 34 wherein the metal salt is selected from the group consisting of carboxylates, carbamates, alkoxides, thiolates, catecholates, oxalates, thiocarboxylates, tropolates, phosphinates, acetylacetonates, iminoacetylacetonates, and bis-iminoacetylacetonates.

36. The catalyst system of claim 34 wherein the metal salt is an Fe(III) salt.

37. The catalyst system of claim 36 wherein the metal salt is Fe(2,4-pentanedionate)₃.

38. The catalyst system of claim 34 wherein the bisarylimine pyridine ligand is selected from ligands having the formula (I) below: 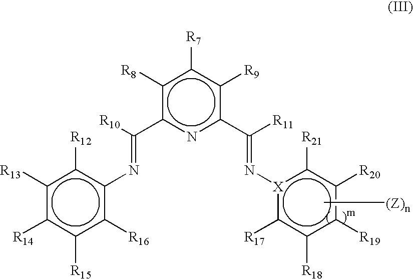
wherein X is carbon or nitrogen,
n is 0 or 1,
m is 0 or 1,
Z is a π-coordinated metal fragment, R₇-R₁₁, R₁₃-R₁₅ and R₁₈-R₂₀ are each, independently, hydrogen, optionally substituted hydrocarbyl, an inert functional group, or any two of R₇-R₉, R₁₃-R₁₅ and R₁₈-R₂₀ vicinal to one another taken together may form a ring; R₁₂ is hydrogen, optionally substituted hydrocarbyl, an inert functional group, or taken together with R₁₃ or R₁₀ to form a ring;
R₁₆ is hydrogen, optionally substituted hydrocarbyl, an inert functional group, or taken together with R₁₅ or R₁₀ to form a ring; R₁₇ is hydrogen, optionally substituted hydrocarbyl, an inert functional group, or taken together with R₁₁ or R₁₈ to form a ring; and R₂₁ is hydrogen, optionally substituted hydrocarbyl, an inert functional group, or taken together with R₁₁ or R₂₀ to form a ring.

39. The catalyst system of claim 34 wherein in the organometallic aluminium compounds of formulae (I) and (II) R¹ is a C₁-C₅ alkyl group; R² is hydrogen or a C₁-C₅ alkyl group; and R³ is a C₁-C₅ alkyl group.

40. The process of claim 34 wherein in the organometallic aluminium compounds of formula (I) or (II) above wherein R⁴, R⁵ and R⁶ are independently selected from hydrogen or a C₁-C₅ alkyl.

41. The process of claim 34 wherein in the organometallic aluminium compounds of formula (I) or (II) above wherein x is 3 and z is 0.

42. The process of claim 34 wherein the organometallic aluminium compound is tris(2,4,4-trimethylpentyl)aluminium.

43. The process of claim 34 wherein the organometallic aluminium compound is tris(2,3-dimethyl-butyl)aluminium.
